SEQUENCE LISTING (1 sequence)

SEQ ID NO: 1 - protein, 10 aa, Artificial Sequence
  Chemically synthesized amino acid sequence
GGLGPAGGCG

(19) United States
(12) Patent Application Publication
(10) Pub. No.: US 2008/0241071 A1
(43) Pub. Date: Oct. 2, 2008

(54) Quantum Dot Probes

(76) Inventors: Jennifer L. West, Houston, TX (US); Rebekah A. Drezek, Houston, TX (US)

Correspondence Address: BAKER BOTTS, LLP, 910 LOUISIANA, HOUSTON, TX, 77002-4995, US

(21) Appl. No.: 12/013,215

(22) Filed: Jan. 11, 2008

Related U.S. Application Data
(63) Continuation of application No. PCT/US2006/027244, filed on Jul. 14, 2006.

(51) Int. Cl. A61K 49/00; G01N 21/64

(52) U.S. Cl. 424/9.6; 436/172

(57) ABSTRACT

Novel nanoparticulate compositions of luminescent probes, as well as methods of using such compositions and systems comprising such compositions are provided. One such composition comprises at least one probe comprising a quantum dot, at least one metal nanoparticle, and at least one tether that is attached to the quantum dot and to the at least one metal nanoparticle. One such method comprises providing at least one such probe, introducing the at least one probe into a subject; and detecting luminescence from the at least one probe in the subject. One such system comprises at least one such probe and a detector capable of detecting luminescence from the quantum dot, wherein the detector is positioned in relation to the at least one probe such that luminescence can be detected.

### CROSS-REFERENCE TO RELATED APPLICATIONS

This application is a continuation of International Application No. PCT/US2006/027244, filed Jul. 14, 2006, which claims the benefit of U.S. Provisional Application No. 60/699,108, filed Jul. 14, 2005.

### STATEMENT OF GOVERNMENT INTEREST

This disclosure was developed with support under Award Number EEC-0118007, awarded by the National Science Foundation. The U.S. government has certain rights in the invention.

### BACKGROUND

The present disclosure, according to certain embodiments, relates generally to nanoparticulate luminescent probes, and more particularly, to nanoparticulate luminescent probes having inherent signal amplification upon interaction with a targeted molecule

Probes may be used to monitor molecular targets and pathways in vivo through optical imaging. Some probes may use organic fluorophores or dyes that are linked to a substrate in a variety of configurations. In certain configurations, fluorophores may be used in close proximity to each other to auto-quench fluorescence. Upon separation of the fluorophores from the substrate, fluorescence may be unquenched.

Another approach is to monitor the loss of fluorescence resonance energy transfer (FRET) between donor/acceptor paired fluorophores. For example, FRET may be used to monitor protease activity in probes that link a fluorophore to a substrate through an enzymatically degradable peptide sequence.

For imaging applications, probes consisting of a fluorescence emitter linked to a non-fluorescent absorber have been developed. These probes, however, lack, among other things, tunability of wavelength. This lack of tunability requires specific pairing between the donor and acceptor. In addition, these probes utilize organic fluorophores, which are often, among other things, inherently unstable in aqueous environments and quickly photobleach, and may be destroyed under a variety of conditions (e.g. exposure to light, change of pH, or a change in temperature). Thus, conventional organic fluorophores are not well suited for applications involving fluorescent quantitation and long term in vivo studies.

### SUMMARY

The present disclosure, according to certain embodiments, relates to probes comprising: a quantum dot, at least one metal nanoparticle, and at least one tether that is attached to the quantum dot and to the at least one metal nanoparticle. Release of metal nanoparticles by disruption of the tether (e.g. through the action of a proteolytic enzyme on its target substrate within the tether) may restore radiative quantum dot luminescence. The quantum dot's luminescence may then be detected and quantified.

In certain embodiments, the present disclosure provides a method of quantifying quantum dot luminescence comprising providing at least one probe comprising a quantum dot, at least one metal nanoparticle, and at least one tether that is attached to the quantum dot and to the at least one metal nanoparticle, introducing the at least one probe into a subject or a sample, and detecting the resulting luminescence of the quantum dot.

In certain embodiments, the present disclosure provides a system comprising at least one probe comprising a quantum dot, at least one metal nanoparticle, and at least one tether that is attached to the quantum dot and to the at least one metal nanoparticle, and a detector capable of detecting luminescence from the quantum dot, wherein the detector is positioned in relation to the at least one probe such that luminescence can be detected.

Other and further features and advantages of the present invention will be readily apparent to those skilled in the art upon a reading of the description of the embodiments that follows.

### FIGURES

A more complete understanding of this disclosure may be acquired by referring to the following description taken in combination with the accompanying figures.

FIG. 1 is a schematic depicting a probe, according to certain embodiments of the present disclosure.

FIG. 2 is an illustration depicting activation of a probe, according to certain embodiments of the present disclosure.

FIG. 3 is a chart demonstrating the reduction in luminescence of a probe, according to certain embodiments of the present disclosure.

FIG. 4 is an emission scan of a probe, according to certain embodiments of the present disclosure.

FIG. 5 is an activation plot of a probe, according to certain embodiments of the present disclosure.

While the present disclosure is susceptible to various modifications and alternative forms, specific example embodiments have been shown in the figures and are described below in more detail. It should be understood, however, that the description of specific example embodiments is not intended to limit the invention to the particular forms disclosed, but on the contrary, this disclosure is to cover all modifications and equivalents as defined by the appended claims.

### DESCRIPTION

The present disclosure, according to certain embodiments, relates generally to nanoparticulate luminescent probes, and more particularly to nanoparticulate luminescent probes having inherent signal amplification upon interaction with a targeted molecule.

In certain embodiments, the present disclosure provides a composition comprising at least one probe that comprises a quantum dot (Qdot, QD), a metal nanoparticle, and a tether, in which the tether is attached to the QD and the metal nanoparticle.

A QD is a semiconductor nanocrystal whose radius is smaller than the bulk exciton Bohr radius. QDs constitutes a class of materials intermediate between molecular and bulk forms of matter. QDs may be formed from inorganic, crystalline semiconductive materials and, among other things, have unique photophysical, photochemical, and nonlinear optical properties arising from quantum size effects. QDs have therefore attracted a great deal of attention for, among other things, their potential applicability in a variety of contexts, which may include, but are not limited to, use as detectable labels in biological applications, and as useful materials in the areas of photocatalysis, charge transfer devices, and analytical chemistry.

In certain embodiments, QDs useful in the compositions and methods of the present disclosure may exhibit a number of unique optical properties due to, among other things, quantum confinement effects. For example, QDs useful in the compositions and methods of the present disclosure may possess strong luminescence, photostability against bleaching and physical environments such as pH and temperature, and optical tunability, overcoming many of the shortcomings apparent with organic fluorophores. These properties may make them suitable for optical imaging and have proven to be useful as in vitro and in vivo biological labels.

As noted above, in certain embodiments, the present disclosure provides a probe that comprises a QD, a metal nanoparticle, and a tether, in which the tether is attached to the QD and the metal nanoparticle. As used herein, the term “attached” may include, but is not limited to, such attachments as covalent binding, adsorption, and physical immobilization. The choice of attachment may depend upon, among other things, the tether and/or metal nanoparticle used and the application for the probe. One of ordinary skill in the art, with the benefit of this disclosure, may recognize additional means of attaching the tether to the QD and/or the nanoparticle. Such attachment methods are considered to be within the spirit of the present invention.

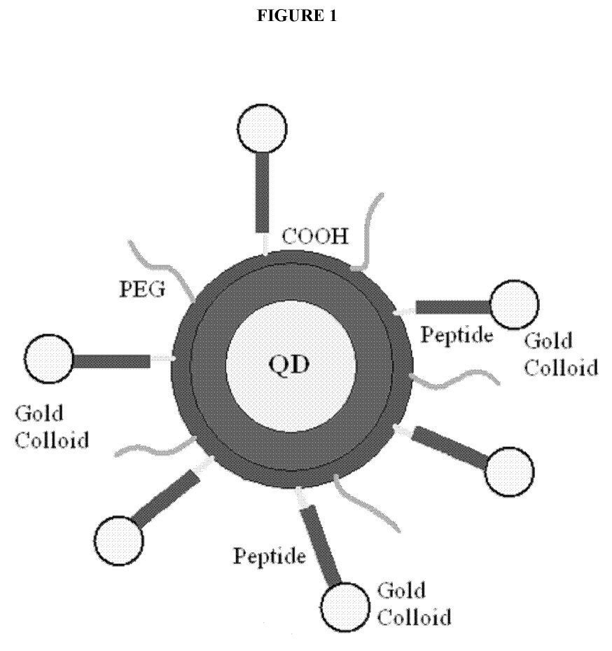

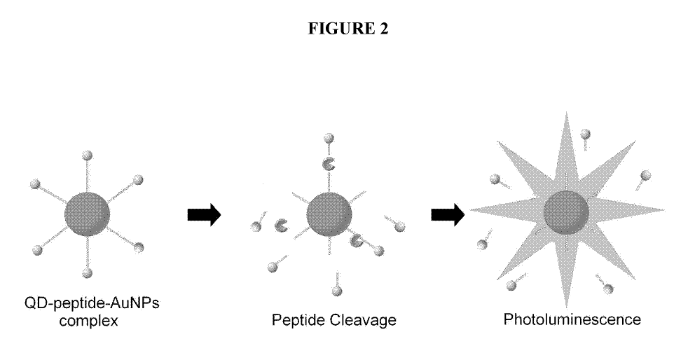

By way of explanation, and not of limitation, in the probes of the present invention, the luminescence of the QD may be non-radiatively suppressed by the metal nanoparticle (e.g., “gold colloid” of FIG. 1) when the QD and metal nanoparticle are attached by the tether (e.g., “peptide” of FIG. 1). Release of metal nanoparticles by disruption of the tether (e.g., “peptide cleavage” illustrated in the middle of FIG. 2) may restore radiative QD luminescence (e.g., “photoluminescence” illustrated in the middle of FIG. 2) through non-radiative energy transfer to make a functional QD probe. Thus, the probe may be tuned by pairing different QDs with different tethers based on, among other things, the desired result and/or the desired application. For example, certain pairings of QDs and tethers may allow for the simultaneous imaging and quantification of numerous targets or activities in vivo. Accordingly, the probes of the present invention may be used in conjunction with optical imaging techniques to monitor specific molecular targets and pathways. Such probes may be useful, among other things, as customizable agents for optical imaging, including, but not limited to, imaging in cancer detection and/or diagnosis. In addition, in certain embodiments, the probes of the present invention may be detected at high resolutions, among other things, because QDs have a small size and are luminescent. The small size and luminescence may be used, for example, to overcome the limited signal-to-background ratio problems often present in conventional targeted imaging.

QDs may be formed from an inner core of one or more first semiconductor materials that optionally may be contained within an overcoating or “shell” of a second semiconductor material. A QD core surrounded by a semiconductor shell is referred to as a “core/shell” QD. In certain embodiments, the optional surrounding shell material will preferably have a bandgap energy that is larger than the bandgap energy of the core material and may be chosen to have an atomic spacing close to that of the core substrate. Suitable semiconductor materials for the core and/or the optional shell include, but are not limited to, the following: materials comprised of a first element selected from Groups 2 and 12 of the Periodic Table of the Elements and a second element selected from Group 16 (e.g., ZnS, ZnSe, ZnTe, CDs, CdSe, CdTe, HgS, HgSe, HgTe, MgS, MgSe, MgTe, CaS, CaSe, CaTe, SrS, SrSe, SrTe, BaS, BaSe, BaTe, and the like); materials comprised of a first element selected from Group 13 of the Periodic Table of the Elements and a second element selected from Group 15 (e.g., GaN, GaP, GaAs, GaSb, InN, InP, InAs, InSb, and the like); materials comprised of a Group 14 element (Ge, Si, and the like); materials such as PbS, PbSe and the like; and alloys and mixtures thereof As used herein, all reference to the Periodic Table of the Elements and groups thereof is to the IUPAC system for numbering element groups, as set forth in the Handbook of Chemistry and Physics, 81st Edition (CRC Press, 2000). QDs may be made using techniques known in the art. See, e.g., U.S. Pat. Nos. 6,048,616; 5,990,479; 5,690,807; 5,505,928; and 5,262,357.

In certain embodiments, QDs useful in the compositions and methods of the present disclosure may absorb a wide spectrum of light and may be physically tuned with emission bandwidths in various wavelengths. See, e.g., Badolato, et al., Science 208:1158-61 (2005). For example, the emission bandwidth may be in the visible spectrum (e.g., from about 3.5 to 7.5 pm), the visible-infrared spectrum (e.g., from about 0.1 to about 0.7 μm), or in the near-infrared spectrum (e.g., from about 0.7 to about 2.5 μm). QDs that emit energy in the visible range may include, but are not limited to, CdS, CdSe, CdTe, ZnSe, ZnTe, GaP, and GaAs. QDs that emit energy in the blue to near-ultraviolet range may include, but are not limited to, ZnS and GaN. QDs that emit energy in the near-infrared range may include, but are not limited to, InP, InAs, InSb, PbS, and PbSe. In certain embodiments, QDs with emission spectra in the near-infrared spectrum may be particularly suited for probes used in certain imaging applications. For example, such QDs may be suited for in vivo imaging, among other things, due to high optical transmissivity of biological tissue in the near-infrared spectrum.

For attachment to certain tethers, the QD may comprise a functional group or attachment moiety. One example of such a QD that has a functional group or attachment moiety is a QD with a carboxylic acid terminated surface, such as those commercially available though, for example, Quantum Dot, Inc., Hayward, Calif. The choice of a functional group or attachment moiety may depend upon, among other things, the tether and/or the application of the resulting composition. One of ordinary skill in the art, with the benefit of this disclosure, will recognize suitable functional groups and/or attachment moieties. Such functional groups and/or attachment moieties are considered within the spirit of the present invention.

Any metal nanoparticle may be used in the compositions and methods of the present invention provided that the metal nanoparticle is capable of attachment to a tether and can quench the QD. Suitable metal nanoparticles may have any shape, including, but not limited to, spherical, elliptical, hollow, and solid, and may have a diameter in the range of about 1 nm to about 1,000 nm (e.g., 2 nm to 50 nm or 2 nm to 20 nm). The choice of a suitable shape may depend upon, among other things, the metal of the metal nanoparticle and the application of the resulting composition. In some examples, suitable metal nanoparticles may be formed from a biocompatible metal, including, but not limited to, gold or silver. One example of a suitable metal nanoparticle is a gold nanoparticle (AuNP), such as a ˜1.4 nm mono-maleimide functionalized AuNP commercially available from Nanoprobes, Yaphank, N.Y. Other suitable AuNPs include, but are not limited to, Au—AuS nanoshells, gold nanorods, and gold nanoshells. One of ordinary skill in the art, with the benefit of this disclosure, may recognize additional metal nanoparticles useful in the compositions and methods of the present disclosure. Such metal nanoparticles are still considered within the spirit of the present invention.

A variety of ratios of metal nanoparticles to quantum dots may be useful in the compositions and methods of the present invention. The choice of a suitable ratio of metal nanoparticles to quantum dots may depend upon, among other things, the application of the resulting composition. In certain embodiments, the compositions and methods of the present invention may use a ratio of metal nanoparticles to quantum dots of about six to about one.

The tether may be any molecule capable of binding a QD and a metal nanoparticle without inducing an adverse and/or undesired effect upon the QD and/or the metal nanoparticle. Suitable tethers may have any length, provided the length does not exceed the energy transfer distance necessary for the metal nanoparticle to at least partially suppress the QD's luminescence. The distance at which energy transfer between two molecules is 50% efficient is known as the Forster radius, which is typically less than about 10 nm. The Forster radius may be determined by, among other things, molecular dipole, quantum yield, refractive index, and spectral overlap. Such determining factors are described in J. R. Lakowicz, Principles of Fluorescence Spectroscopy, Kluwer Academic/Plenum Publisher, New York (1999). Examples of molecules suitable for use as a tether may include, but are not limited to, a molecule having at least one substrate for a proteolytic enzyme, a peptide, a nucleic acid (e.g., DNA or RNA), a molecule having a hydrolyzable ester bond, and an N-isopropylacrylamide (NIPAAm) molecule. The particular tether chosen may depend on, among other things, the metal nanoparticle and/or the desired application and/or target for the composition.

In certain embodiments, the compositions of the present invention may comprise a tether comprising a nucleic acid. Such compositions may be capable of, among other things, detecting DNAses or RNAses. In such compositions, luminescence may be correlated with nucleic acid hybridization (e.g., as in a gene chip).

In certain embodiments, the compositions of the present invention may comprise a tether having a hydrolyzable ester bond. Such compositions may be suitable for use as a pH sensor, as ester bonds may be pH sensitive. Accordingly, hydrolysis of the ester bond in such compositions may result in luminescence of the QD, which may be correlated to pH.

In certain embodiments, the compositions of the present invention may comprise a tether comprising a NIPAAm molecule. Such compositions may be suitable for use as a temperature sensor. By way of explanation, and not of limitation, the conformation of NIPAAm may be dependent on temperature, and NIPAAm can be synthesized across a range of temperature sensitivities. Accordingly, when such compositions of the present disclosure comprising a tether comprising a NIPAAm molecule are used, the probe may be capable of luminescence when the temperature is sufficient to disrupt the NIPAAm molecule. In certain embodiments, such compositions may be used in conjunction with microelectromechanical devices (MEMS) with very broad sensitivity ranges.

In another embodiment, the compositions of the present invention may comprise a tether comprising a substrate for a proteolytic enzyme. Such compositions may be suitable for detection of protease activity. In certain embodiments, the compositions of the present invention may comprise a tether comprising a proteolytically sensitive tether, for example, a peptide sequence that can be degraded by a protease, or a synthetic substrate for a proteolytic enzyme (FIG. 2). Proteolytic activity is known to impact a wide range of biological phenomena, including, but not limited to, development, cancer growth and metastasis, wound healing, cell migration, leukocyte extravasation, and degenerative diseases and conditions (e.g. arthritis). Accordingly, compositions of the present invention comprising a tether comprising a substrate for a proteolytic enzyme may be suitable for use in detecting, quantifying, and/or localizing proteolytic activity to, among other things, better diagnose and/or treat precancers, cancers, and degenerative conditions, as well as to design new therapeutics to combat these ailments. In particular, such compositions may be useful in assessing the metastatic potential of a tumor during imaging and diagnosis.

A variety of peptide sequences susceptible to proteolytic cleavage are available. This allows wide applicability of such probes for applications including, but not limited to, imaging proteolytic activity of cells and assessing the metastatic potential of cancerous lesions due to, inter alia, optical tunability and/or adjustable peptide sequences. In certain embodiments, the small size of the compositions of the present invention, among other things, may enable detection of single-cell precancers and single-cell cancers throughout a subject. In one example, a library of different wavelength emitting probes (each probe using spectrally distinct QDs) may be formed, in which the probes have different enzyme specificities to allow for simultaneous imaging from many different proteases. In this way, the detection of multiple types of proteases in the same preparation may be possible, for example, by providing quantifiable data about several different proteases at once. In another example, the compositions of the present invention may be attached to a biomimetic scaffold to follow, for example, proteolytic activity of migrating cells over time.

Suitable peptide sequences may be synthesized using standard techniques, for example, Fmoc (9-flourenylmethloxycarbonyl) solid phase peptide synthesis. Once synthesized, the tether may be attached to a QD. In certain embodiments, the tether may comprise a peptide, the tether may be attached to the QD by covalently attaching the N-terminus of the peptide to one or more carboxylic acid residues on the QD surface under conditions specific for amine reactivity and allowing an amide to form. In certain embodiments, a thiol on the peptide (for example, from a cysteine residue in the peptide sequence) may be reacted with a metal nanoparticle, such as an AuNP, to complete the composition. In certain embodiments, such attachments may be readily achieved under most conditions, because the sulfur-gold bond is often spontaneously formed. Alternatively, NANOGOLD™ monomaleimide (Nanoprobes, Inc., Yaphank, N.Y.) may be reacted with the peptide thiol, with the NANOGOLD™ serving as the AuNP.

In certain embodiments, the compositions of the present invention may be treated, inter alia, to increase biocompatibility. Such a treatment may be useful when the compositions of the present invention are used in, for example, therapeutic applications. For example, the QDs and/or metal nanoparticles may be coated with a biocompatible moiety, for example, polyethylene glycol (PEG) (FIG. 1). One of ordinary skill in the art, with the benefit of this disclosure, may recognize other suitable treatments. Such treatments are still considered within the spirit of the present invention.

In certain embodiments, a linkers may be used between the QD and tether or between the tether and a metal nanoparticle, or both. Such linkers may be useful, among other things, to maximize quenching and/or enzymatic accessibility. The choice of a suitable linker may depend upon, among other things, the tether and/or nanoparticle chosen or the application of the resulting composition. One example of a suitable linker is a PEG linker. One of ordinary skill in the art, with the benefit of this disclosure, may recognize additional linkers which may be suitable for use in the compositions and methods of the present disclosure. Such linkers are still considered within the spirit of the present invention.

In certain embodiments, the probes may be conjugated with at least one additional molecule selected from the group consisting of an antibody, an antigen, and streptavidin.

In certain embodiments, the compositions and methods of the present invention may be used in therapeutic or diagnostic applications. For therapeutic applications, the compositions of the present invention may be administered (e.g., by intravenous injection) to a subject (e.g., a human or other mammal), and any luminescence from the composition may be detected. For diagnostic applications, the compositions of the present invention may be applied to a sample (e.g., a biological sample), and any luminescence from the composition may be detected.

In certain embodiments, the present disclosure provides a system comprising a composition of the present invention and a detector capable of detecting luminescence from a QD. To detect luminescence, the detector should be positioned in relation to the probe such that luminescence can be detected, and the detector should be adapted to detect luminescence. One example of a suitable detector is a fluorimeter. In certain embodiments, the system of the present invention may further comprise a source of electromagnetic radiation, for example, an excitation monochromator. An excitation monochromator may be used, among other things, to excide the probe at a specific wavelength to ensure the quality of emission detection.

To facilitate a better understanding of the present disclosure, the following examples of specific embodiments are given. In no way should the following examples be read to limit or define the entire scope of the invention.

### EXAMPLES

Quantum dot Synthesis

Core/shell structured CdSe/CdS QDs were synthesized as described in J. J. Li, et al., Am. Chem. Soc. 125:12567-75. Poly(ethylene glycol) (PEG, 750 Da) was used to increase the water-solubility and stability of the QDs. The resulting QDs are carboxylate-terminated with a peak emission at 620 nm.

Peptide Synthesis

A collagenase-degradable specific peptide sequence (GGLGPAGGCG) was used. The GGLGPAGGCG degradable peptide sequence was synthesized using Fmoc solid phase peptide synthesis (Applied Biosystems, Inc., Foster City, Calif.). Cleavage from the polystyrene resin was effected with 95% trifluoroacetic acid, 2.5% water, and 2.5% triusopropylsilane. The cleaved peptide was precipitated in ether followed by dialysis against MilliQ water (Milli-Q Gradient, Millipore, Billerica, Mass.). The peptide was lyophilized and stored at −20° C. until use.

Conjugation Reaction

QDs (2 nmol) in deionized water were activated with EDC and sulfo-NHS (Pierce, Rockford, Ill.) to form an active ester leaving group. The N-terminus of the synthesized peptide was then covalently linked to the QDs at the active ester site to form an amide. Activation of the C-terminus of the peptide was prevented by reacting residual EDC with β-mercaptoethanol prior to peptide addition. During the coupling reaction, peptide was added in a 30-fold molar excess to ensure sufficient coupling onto the QD. The reaction was allowed to proceed overnight in the dark at room temperature. The solution was then dialyzed with 5,000 MWCO cellulose ester membrane (Spectrum Laboratories, Houston, Tex.) to remove any unreacted peptide or byproducts. Following dialysis, the solution was split into two aliquots. One aliquot was reacted with gold nanoparticles while the other aliquot served as control. The control underwent identical steps as the conjugate except it was reacted with equal volumetric amounts of deionized water rather than gold nanoparticles. The quantum dot-peptide conjugate was concentrated using a 50,000 MWCO Vivaspin Ultrafiltration concentrator (Vivascience A G, Hannover, Germany) and centrifuged at 2,000×g for 20 min. The purified QD-peptide conjugate was resuspended to 400 μL of deionized water.

Mono-maleimide functionalized AuNPs (1.4 nm; Nanoprobes, Yaphank, N.Y.) were covalently linked to the sulfhydryl group on the cysteine residue of the QD-peptide conjugate at a ratio of 6:1 AuNP:QD. A centrifuge filter (Vivaspin 6 MWCO 50,000) was then used to remove unbound AuNPs and the probe was resuspended in sterile HEPES-buffered saline (HBS: 135 mM NaCl, 5 mM KCl, 1 mM MgSO₄, 1.8 mM CaCl₂, 10 mM HEPES, pH 7.4). Luminescence measurements were made on the control and conjugate to compare quenching of the quantum dots by the gold nanoparticles.

Activation of Probe

Following initial luminescence measurements, collagenase Type XI (Sigma-Aldrich, St. Louis, Mo.) was added to the probes at a final concentration of 0.2 mg/mL. Control samples (QD probe without collagenase) were monitored simultaneously. Extinction measurements were made of varying concentrations of collagenase in HBS to examine effects on turbidity which may affect luminescence measurements. Studies demonstrated minimal effects on turbidity at wavelengths >450 nm for concentrations of 0.2 mg/mL and lower. Fluorescence measurements of collagenase (0.2 mg/mL) in HBS (n=3) were used to subtract autofluorescence from collagenase in all probe samples containing collagenase. QD probes with collagenase were incubated at room temperature. Photoluminescence was measured to observe unquenching of QDs in HBS over time. Studies were limited to less than 48 hours due to the progressive loss of collagenase bioactivity in solution.

Spectroscopy Measurements

All measurements were performed at room temperature with a 500 μL stoppered quartz cuvette to prevent evaporation (Stama Cells Inc., Atascadero, Calif.) on a Horiba Jobin Yvon SPEX FL3-22 Fluorimeter (Edison, N.J.) with dual excitation and emission monochromators. Time-integrated photoluminescence was measured before and after conjugation to the AuNP to observe quenching of QD probes. Photoluminescence measurements were also taken over time to observe proteolytic activation of the probe. Baseline values were taken for all measurements of QD probes in deionized water and HBS. QDs were excited at 360 nm, and emission scans measured from 400-700 nm. Bandpass slits and integration time were set to 3 nm/3 nm and 100 ms, respectively on the fluorimeter. All values were normalized over time to a rhodamine 6G standard to avoid any artifacts that could arise from possible lamp fluctuations. Extinction measurements from 200-800 nm were also acquired for each sample on a Varian Cary 50 Bio spectrophotometer (Walnut Creek, Calif.).

Results

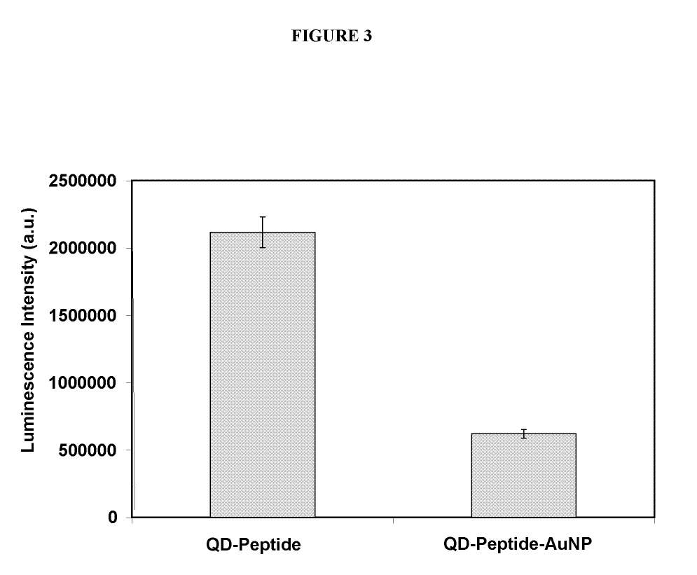

Results of quantum dot quenching by AuNPs are shown in FIG. 3. All measurements reported consist of a sample size n=3. For a 6:1 ratio of AuNP:QD, QD-peptide-AuNP luminescence indicates a 71% quenching of signal compared to unreacted QD-peptide. In addition, initial measurements at t=0 hours indicate no statistical difference between probe without collagenase and probe with collagenase after subtraction of collagenase autofluorescence.

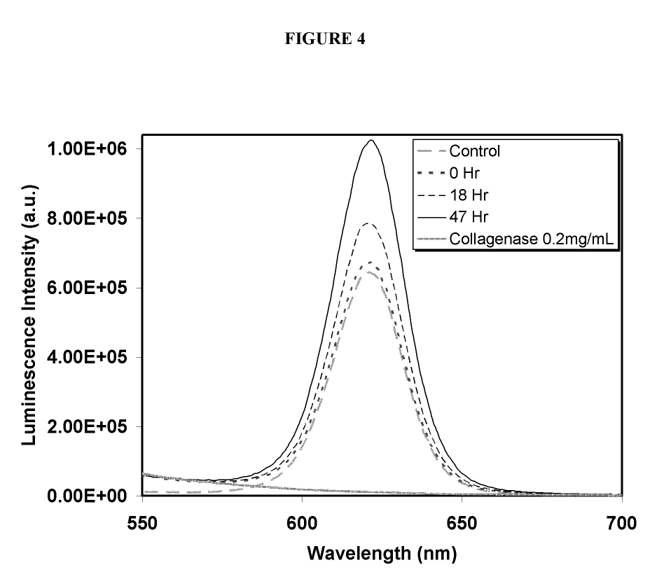

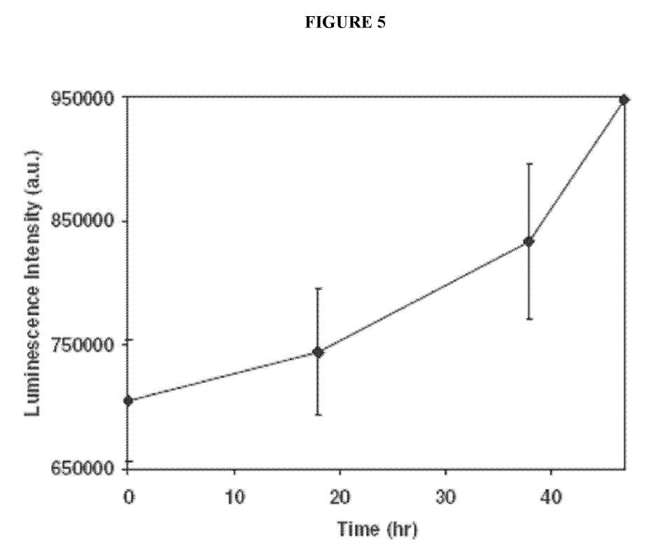

Luminescence of each sample was measured at the following time points: 0, 18, 38, and 47 hours. FIG. 4 illustrates the spectral profile of increased QD luminescence over time. At collagenase concentrations of 0.2 mg/mL, there is minimal collagenase autofluorescence past 550 nm. A rise over time in luminescence of the probe with collagenase is observed in FIG. 5. At 47 hours, an average luminescence rise of 52% is observed. The control group containing inactivated probe (QD probe without collagenase) in buffer solution indicated statistically insignificant changes in luminescence during the same time intervals.

While embodiments of this disclosure have been depicted, described, and are defined by reference to embodiments of the disclosure, such references do not imply a limitation on the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

What is claimed is:

1. A composition comprising:
at least one probe comprising:
a quantum dot;
at least one metal nanoparticle; and
at least one tether that is attached to the quantum dot and to the at least one metal nanoparticle.

2. The composition of claim 1, wherein the at least one probe comprises at least one metal nanoparticle comprising a nanoshell or a nanorod.

3. The composition of claim 1, wherein the at least one probe comprises at least one metal nanoparticle comprising a gold nanoparticle.

4. The composition of claim 1, wherein the at least one probe comprises a quantum dot having an emission bandwidth in a range selected from the group consisting of:
the visible spectrum, the visible-infrared spectrum, the near-infrared spectrum, and the infrared spectrum.

5. The composition of claim 1, wherein the at least one probe comprises a quantum dot comprising a functional group, an attachment moiety, and any combination thereof

6. The composition of claim 1, wherein the at least one probe comprises at least tether comprising at least one molecule selected from the group consisting of: a molecule having at least one substrate for a proteolytic enzyme, a peptide, a nucleic acid, a DNA molecule, an RNA molecule, a molecule having a hydrolyzable ester bond, and an N-isopropylacrylamide molecule.

7. The composition of claim 1, wherein the at least one probe further comprises a polyethylene glycol molecule, wherein the polyethylene glycol molecule is attached to one or more of the quantum dot, the metal nanoparticle, and the tether.

8. The composition of claim 1, wherein the at least one probe further comprises at least one conjugate selected from the group consisting of: an antibody, an antigen, and streptavidin.

9. The composition of claim 1, wherein the ratio of the metal nanoparticles to the quantum dot is about six to about one.

10. The composition of claim 1, wherein the at least one metal nanoparticle has a diameter in the range of about 1 nm to about 1,000 nm.

11. The composition of claim 1, further comprising a biocompatible moiety.

12. A method comprising:
providing at least one probe comprising:
a quantum dot;
at least one metal nanoparticle; and
at least one tether that is attached to the quantum dot and to the at least one metal nanoparticle;
introducing the at least one probe into a subject; and
detecting luminescence from the at least one probe in the subject.

13. The method of claim 12, wherein the at least one probe comprises at least one tether comprising at least one molecule selected from the group consisting of: a molecule having at least one substrate for a proteolytic enzyme, a peptide, a nucleic acid, a DNA molecule, an RNA molecule, a molecule having a hydrolyzable ester bond, and an N-isopropylacrylamide molecule.

14. The method of claim 12, wherein the at least one probe is attached to a biomimetic scaffold.

15. A method comprising:
providing at least one probe comprising:
a quantum dot;
at least one metal nanoparticle; and
at least one tether that is attached to the quantum dot and to the at least one metal nanoparticle;
introducing the at least one probe into a sample; and
detecting luminescence from the at least one probe in the sample.

16. The method of claim 15, wherein the at least one probe comprises at least one tether comprising at least one molecule selected from the group consisting of: a molecule having at least one substrate for a proteolytic enzyme, a peptide, a nucleic acid, a DNA molecule, an RNA molecule, a molecule having a hydrolyzable ester bond, and an N-isopropylacrylamide molecule.

17. The method of claim 15, wherein the at least one probe is attached to a biomimetic scaffold.

18. A system comprising:
at least one probe comprising:
a quantum dot;
at least one metal nanoparticle; and
at least one tether that is attached to the quantum dot and to the at least one metal nanoparticle; and
a detector capable of detecting luminescence from the quantum dot, wherein the detector is positioned in relation to the at least one probe such that luminescence can be detected.

19. The system of claim 18, wherein the tether comprises at least one molecule chosen from a molecule that is a substrate for a proteolytic enzyme, a peptide, a nucleic acid, a DNA molecule, an RNA molecule, a molecule having a hydrolyzable ester bond, and an N-isopropylacrylamide molecule.

20. The system of claim 18, wherein the detector is a fluorimeter.

21. The system of claim 18 further comprising a source of electromagnetic radiation.

22. The system of claim 18 further comprising an excitation monochromator.
